(19) United States
(12) Patent Application Publication
(10) Pub. No.: US 2012/0263726 A1
(43) Pub. Date: Oct. 18, 2012

(54) Pharmaceutical composition and method of inhibiting of production or amplifying of elimination of P24 protein

(76) Inventors: Oleg Iliich Epshtein, Moscow (RU); Sergey Alexandrovich Tarasov, Golitsyno (RU)

(21) Appl. No.: 13/135,897

(22) Filed: Jul. 15, 2011

(30) Foreign Application Priority Data
Aug. 6, 2010 (RU) 2010133046

(51) Int. Cl. A61K 39/395; A61P 31/18; C07K 16/28

(52) U.S. Cl. 424/139.1; 530/387.9

(57) ABSTRACT

The present invention relates to a pharmaceutical composition, comprising an activated-potentiated form of an antibody to CD4 receptor, and method of inhibiting of production or amplifying of elimination of P24 protein.

### FIELD

The present invention relates to a pharmaceutical composition and method of inhibiting of production or amplifying of elimination of P24 protein.

### BACKGROUND

The invention relates to the area of medicine and can be used to inhibit the production or amplify the elimination of protein P24.

P24 protein detected in the human body is known in the art (see Papadopulos-Eleopulos E, Turner V F, Papadimitriou J M. Is a positive western blot proof of HIV infection? Biotechnology (NY). 1993 June; 11 (6): 696-707).

Use of antibodies to CD4 molecules conjugated with anti-chemokine receptors for treatment of HIV-1 is known in the art (WO 01/43779 A2, A61K 47/48, 2001). However, experimental data about therapeutic efficacy of this drug in mentioned source are missing.

The therapeutic effect of an extremely diluted form (or ultra-low form) of antibodies potentized by homeopathic technology (activated-potentiated form) has been discovered by Dr. Oleg I. Epshtein. For example, U.S. Pat. No. 7,582,294 discloses a medicament for treating Benign Prostatic Hyperplasia or prostatitis by administration of a homeopathically activated form of antibodies to prostate specific antigen (PSA). Ultra-low doses of antibodies to gamma interferon have been shown to be useful in the treatment and prophylaxis of treating diseases of viral etiology. See U.S. Pat. No. 7,572,441, which is incorporated herein by reference in its entirety.

CD4 (cluster of differentiation 4), or CD4 receptor of immune cells, is a glycoprotein expressed on the surface of immune cells such as leucocytes, T helper cells, regulatory T cells, monocytes, macrophages, and dendrytic cells. Like many cell surface receptors/markers, CD4 is a member of the immunoglobulin superfamily. CD4 is a co-receptor that assists the T cell receptor (TCR) with an antigen-presenting cell. Using its portion that resides inside the T cell, CD4 amplifies the signal generated by the TCR by recruiting an enzyme, known as lymphocyte-specific protein tyrosine kinase, which is essential for activating many molecules involved in the signaling cascade of an activated T cell. CD4 also interacts directly with MHC class II molecules on the surface of the antigen-presenting cell using its extracellular domain. HIV-1 uses CD4 to gain entry into host T-cells and achieves this by binding of the viral envelope protein known as gp120 to CD4. The binding to CD4 creates a shift in the conformation of gp120 allowing HIV-1 to bind to a co-receptor expressed on the host cell. These co-receptors are chemokine receptors CCR5 or CXCR4, which of these co-receptor is used during infection is dependent on whether the virus is infecting a macrophage or T-helper cent. Following a structural change in another viral protein (gp41), HIV inserts a fusion peptide into the host cell that allows the outer membrane of the virus to fuse with the cell membrane. See Miceli M C, Parnes J R (1993). “Role of CD4 and CD8 in T cell activation and differentiation”. Adv. Immunol. 53: 59-122.

The present invention is directed to a pharmaceutical composition and method of inhibiting of production or amplifying of elimination of P24 protein.

The solution to the existing problem is presented in form of a pharmaceutical composition for inhibiting of production or amplifying of elimination of P24 protein, which comprises activated-potentiated form of antibodies to CD4 receptor.

### SUMMARY

In one aspect, the invention provides a pharmaceutical composition comprising an activated-potentiated form of an antibody to CD4 receptor. In an embodiment, the pharmaceutical composition further comprises a solid carrier, wherein said activated-potentiated form of an antibody to CD4 receptor is impregnated onto said solid carrier. In a variant, the pharmaceutical composition is in the form of a tablet.

Preferably, the pharmaceutical composition including said activated-potentiated form of an antibody to CD4 receptor is in the form of a mixture of C12, C30, and C200 homeopathic dilutions. It is specifically contemplated that said mixture of C12, C30, and C200 homeopathic dilutions is impregnated onto a solid carrier.

Preferably, the pharmaceutical composition including said activated-potentiated form of an antibody to CD4 receptor is in the form of a mixture of C12, C30, and C50 homeopathic dilutions. It is specifically contemplated that said mixture of C12, C30, and C50 homeopathic dilutions is impregnated onto a solid carrier.

The activated-potentiated form of an antibody to CD4 receptor may be a monoclonal, polyclonal or natural antibody. It is specifically contemplated that the activated-potentiated form of an antibody to CD4 receptor is a polyclonal antibody. The invention provides activated-potentiated forms of antibodies to antigen(s) having sequences described in the specification and claimed in the appended claims.

In a variant, the pharmaceutical composition includes activated-potentiated form of an antibody to CD4 receptor prepared by successive centesimal dilutions coupled with shaking of every dilution. Vertical shaking is specifically contemplated.

In another aspect, the invention provides a method of inhibiting of production or amplifying of elimination of P24 protein, said method comprising administering an activated-potentiated form of an antibody to CD4 receptor thereby inhibiting production or amplifying elimination of P24 protein. Preferably, the activated-potentiated form of an antibody to CD4 receptor is administered in the form of pharmaceutical composition.

In an embodiment, the pharmaceutical composition is administered in the form of a solid oral dosage form which comprises a pharmaceutically acceptable carrier and said activated-potentiated form of an antibody to CD4 receptor impregnated onto said carrier. In a variant, said solid oral dosage form is a tablet. Variants and embodiments are provided.

In accordance with the method aspect of the invention, the pharmaceutical composition may be administered in one to two unit dosage forms, each of the dosage form being administered from once daily to four times daily. In a variant, the pharmaceutical composition is administered twice daily, each administration consisting of two oral dosage forms. In a variant, the pharmaceutical composition is administered in one to two unit dosage forms, each of the dosage forms being administered twice daily. All variants and embodiments described with respect to the composition aspect of the invention may be used with the method aspect of the invention.

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab')₂, Fab', and the like. The singular “antibody” includes plural “antibodies.”

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³⁰ and 100⁵⁰ times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term ‘activated-potentiated” form used in the claims.

In other words, an antibody is in the “activated-potentiated” or “potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well-accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated-potentiated” form of the antibodies described herein is described in more detail below.

There has been a considerable amount of controversy regarding homeopathic treatment of human subjects. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity. The experiments provided further below provide evidence of biological activity in such models; it is associated by higher antiviral and, possibly, immunotropic action, intensification of activation of CD4 lymphocytes and enrichment of number of receptors on the surface of CD4 cells.

Also, the claimed “activated-potentiated” form of antibody encompasses only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated” form of the antibodies of the present invention is not attributable to the initial molecular is the “activated-potentiated” form of antibody in liquid or solid form in which the concentration of the molecular form of the antibody is below the Avogadro number. In the pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

The present invention provides a pharmaceutical composition that includes activated-potentiated form of antibodies to CD4 receptor, prepared according to the homeopathic technology of potentiation by repeated, consistent dilution and intermediate external action of shaking as described in more detail herein below. The pharmaceutical composition of the invention is particularly useful in inhibiting of production or amplifying of elimination of P24 protein. As shown in the Examples, the pharmaceutical composition of the invention possesses unexpected therapeutic effect, which manifest itself in particular therapeutic effectiveness in treatment of diseases associated with increase in production of P24 protein.

The pharmaceutical composition of the invention expands the arsenal of preparations available for inhibiting of production or amplifying of elimination of P24 protein.

The pharmaceutical composition in accordance with this aspect of the invention may be in the liquid form or in solid form. Activated potentiated form of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R.—2005—Vol. 14.—N 1-2. P.33-55, both incorporated herein by reference.

Monoclonal antibodies may be obtained, e.g., by means of hybridoma technology. The initial stage of the process includes immunization based on the principles already developed in the course of polyclonal antisera preparation. Further antibodies, 30 years after” by Laffly E., Sodoyer R.—2005—Vol. 14.—N 1-2. P.33-55, both incorporated herein by reference.

Monoclonal antibodies may be obtained, e.g., by means of hybridoma technology. The initial stage of the process includes immunization based on the principles already developed in the course of polyclonal antisera preparation. Further stages of work involve the production of hybrid cells generating clones of antibodies with identical specificity. Their separate isolation is performed using the same methods as in the case of polyclonal antisera preparation.

Polyclonal antibodies may be obtained via active immunization of animals. For this purpose, for example, suitable animals (e.g. rabbits) receive a series of injections of the appropriate antigen (CD4 receptor). The animals' immune system generates corresponding antibodies, which are collected from the animals in a known manner. This procedure enables preparation of a monospecific antibody-rich serum.

If desired, the serum containing antibodies may be purified, for example by using affine chromatography, fractionation by salt precipitation, or ion-exchange chromatography. The resulting purified, antibody-enriched serum may be used as a starting material for the preparation of the activated-potentiated form of the antibodies. The preferred concentration of the resulting initial solution of antibody in the solvent, preferably water or a water-ethyl alcohol mixture, ranges from about 0.5 to about 5.0 mg/ml.

The preferred procedure for preparing each component of the combination drug according to the present invention is the use of the mixture of three aqueous-alcohol dilutions of the primary matrix solution of antibodies diluted 100¹², 100³⁰ and 100⁵⁰ times, respectively, which is equivalent to centesimal homeopathic dilutions C12, C30, and C50 or diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In a preferred embodiment, the starting material for the preparation of the activated potentiated form that comprise the pharmaceutical composition of the invention is polyclonal, animal-raised antibody to the corresponding antigen, namely, CD4 receptor. To obtain the activated-potentiated form of polyclonal antibodies to CD4 receptor, the desired antigen may be injected as immunogen into a laboratory animal, preferably, rabbits. Polyclonal antibodies to CD4 receptor may be obtained using the whole molecule of human CD4 receptor of the following sequence:

```
SEQ ID NO: 1
Met Asn Arg Gly Val Pro Phe Arg His Leu Leu Leu Val Leu Gln
  1 5 10 15
Leu Ala Leu Leu Pro Ala Ala Thr Gln Gly Lys Lys Val Val Leu
  16 20 25 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
```

The polyclonal antibodies to CD4 receptor can be obtained using a polypeptide fragment of CD4 receptor chosen, for example, from the following amino-acid sequences:

```
SEQ ID NO: 2
                                                                        Gly Lys Lys Val Val Leu
                                                                          26 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ ID NO: 3
                                                Ile Gly Leu Gly Ile Phe Phe Cys Val
                                                412 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ ID NO: 4
                                                                        Gly Lys Lys Val Val Leu
                                                                          26 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
SEQ ID NO: 5
                                                                                                                Asp
  91 95 100 105
Thr Tyr Ile Cys Glu Val Glu Asp Gln Lys Glu Glu Val Gln
106 110 115 119
SEQ ID NO: 6
                                                                        Lys Glu Glu Val Gln Leu
                                                                        115 120
Leu Val Phe Gly Leu Thr Ala Asn Ser Asp Thr His Leu Leu Gln
121 125 130 135
Gly Gln Ser Leu
136 139
```

The exemplary procedure for preparation of the starting polyclonal antibodies to CD4 receptor may be described as follows. In 7-9 days before blood sampling, 1-3 intravenous injections of the desired antigen are made to the rabbits to increase the level of polyclonal antibodies in the rabbit blood stream. Upon immunization, blood samples are taken to test the antibody level. Typically, the maximum level of immune reaction of the soluble antigen is achieved within 40 to 60 days after the first injection of the antigen. Upon completion of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in a 50 ml centrifuge tube. Product clots formed on the tube sides are removed with a wooden spatula, and a rod is placed into the clot in the tube center. The blood is then placed in a refrigerator for one night at the temperature of about 40° C. On the following day, the clot on the spatula is removed, and the remaining liquid is centrifuged for 10 min at 13,000 rotations per minute. Supernatant fluid is the target antiserum. The obtained antiserum is typically yellow. 20% of NaN₃ (weight concentration) is added in the antiserum to a final concentration of 0.02% and stored before use in frozen state at the temperature of −20° C. or without NaN₃ at the temperature of −70° C. To separate the target antibodies to gamma interferon from the antiserum, the following solid phase absorption sequence is suitable:

10 ml of the antiserum of rabbits is diluted twofold with 0.15 M NaCl, after which 6.26g Na₂SO₄ is added, mixed and incubated for 12-16 hours at 4° C. The sediment is removed by centrifugation, diluted in 10 ml of phosphate buffer and dialyzed against the same buffer during one night at ambient temperature. After the sediment is removed, the solution is applied to a DEAE-cellulose column balanced by phosphate buffer. The antibody fraction is determined by measuring the optical density of the eluate at 280 nm.

The isolated crude antibodies are purified using affine chromatography method by attaching the obtained antibodies to CD4 antigen located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to CD4 receptor is 0.5 to 5.0 mg/ml, preferably, 2.0 to 3.0 mg/ml.

The activated-potentiated form of an antibody to CD4 receptor may be prepared from an initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution) of a neutral solvent, starting with a concentration of the initial solution of antibody in the solvent, preferably, water or a water-ethyl alcohol mixture, in the range from about 0.5 to about 5.0 mg/ml, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to CD4 receptor with the concentration of 3.0 mg/ml is diluted in 99 parts of neutral aqueous or aqueous-alcohol solvent (preferably, 15%-ethyl alcohol) and then vertically shaked many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to gamma interferon with the concentration of 3.0 mg/ml in 99 parts of a neutral solvent in different containers, which is equivalent to the centesimal homeopathic dilution C12. Similar procedures with the relevant dilution factor are performed to obtain dilutions C30, C50 and C 200. The intermediate dilutions may be tested in a desired biological model to check activity. The preferred activated-potentiated form for the composition of the invention are a mixture of C12, C30, and C50 dilutions or C12, C30 and C200 dilutions. When using the mixture of various homeopathic dilutions (primarily centesimal) of the active substance as biologically active liquid component, each component of the composition (e.g., C12, C30, C50, C200) is prepared separately according to the above-described procedure until the next-to-last dilution is obtained (e.g., until C11, C29, and C199 respectively), and then one part of each component is added in one container according to the mixture composition and mixed with the required quantity of the solvent (e.g. with 97 parts for centesimal dilution).

It is possible to use the active substance as mixture of various homeopathic dilutions, e.g. decimal and/or centesimal (D20, C30, C100 or C12, C30, C50 or C12, C30, C200, etc.), the efficiency of which is determined experimentally by testing the dilution in a suitable biological model, for example, in models described in the examples herein.

In the course of potentiation and concentration decrease, the vertical shaking may be substituted for external exposure to ultrasound, electromagnetic field or any similar external impact procedure accepted in the homeopathic art.

Preferably, the pharmaceutical composition of the invention may be in the form of a liquid or in the solid unit dosage form. The preferred liquid carrier is water or water-ethyl alcohol mixture.

The solid unit dosage form of the pharmaceutical composition of the invention may be prepared by impregnating a solid, pharmaceutically acceptable carrier with the mixture of the activated potentiated form aqueous or aqueous-alcohol solutions of active component. Alternatively, the carrier may be impregnated consecutively with each requisite dilution. Both orders of impregnation are acceptable.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies CD4 receptor. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono- olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose, magnesium stearate and citric acid.

To prepare the solid oral form, 100-300 μm granules of lactose are impregnated with aqueous or aqueous-alcoholic solutions of the activated-potentiated form of antibodies to CD4 receptor in the ratio of 1 kg of antibody solution to 5 or 10 kg of lactose (1:5 to 1:10). To effect impregnation, the lactose granules are exposed to saturation irrigation in the fluidized boiling bed in a boiling bed plant (e.g. “Hüttlin Pilotlab” by Hüttlin GmbH) with subsequent drying via heated air flow at a temperature below 40° C. The estimated quantity of the dried granules (10 to 34 weight parts) saturated with the activated potentiated form of antibodies is placed in the mixer, and mixed with 25 to 45 weight parts of “non-saturated” pure lactose (used for the purposes of cost reduction and simplification and acceleration of the technological process without decreasing the treatment efficiency), together with 0.1 to 1 weight parts of magnesium stearate, and 3 to 10 weight parts of microcrystalline cellulose. The obtained tablet mass is uniformly mixed, and tableted by direct dry pressing (e.g., in a Korsch—XL 400 tablet press) to form 150 to 500 mg round pills, preferably, 300 mg. After tableting, 300 mg pills are obtained that are saturated with aqueous-alcohol solution (3.0-6.0 mg/pill) of the activated-potentiated form of antibodies to CD4 receptor in the form of a mixture of centesimal homeopathic dilutions C12, C30, and C50 or a mixture of centesimal homeopathic dilutions C12, C30 and C200.

While the invention is not limited to any specific theory, it is believed that the activated potentiated form of the antibodies described herein do not contain the molecular form of the antibody in an amount sufficient to have biological activity attributed to such molecular form. The biological activity of the combination drug (pharmaceutical composition) of the invention is amply demonstrated in the appended examples.

Preferably, the combination of the invention is administered from once daily to four times daily, preferably twice daily, each administration including one or two combination unit dosage forms.

The invention is further illustrated with reference to the appended non-limiting examples.

### EXAMPLES

### Example 1

The assessment of efficacy of inhibition of production or amplification of elimination of the protein P24 by ultra low-dose of rabbit polyclonal antibodies to CD4 receptor (a mixture of homoeopathic dilutions C12+C30+C50) (ULD Ab CD4)), was carried out using human peripheral blood mononuclear cells infected with the strain HIV-1LAI in vitro.

Human peripheral blood mononuclear cells were isolated from blood of a seronegative healthy donor by centrifugation on a Ficoll-Hypaque density gradient. The cells were stimulated for 3 days with 1 μg/mL of phytohemagglutinin P and 5 IU/mL of recombinant human interleukin-2.

In order to assess the efficacy of inhibition of production or amplification of elimination of the protein P24 the products were placed in a well containing 100 μL of activated mononuclears 24 hours before or 15 min after cell infection with the strain HIV-1-LAI at the dose of 100 TCID50 (50 μL inoculum of the strain HIV-1-LAI). Before adding to a well, ULD Ab CD4 (12.5 μL) were mixed with RPMI1640 medium (DIFCO) to achieve a final probe volume of 50 μL

The supernatant fluids were collected on day 7 after infection of cells. The products' activity was measured by the inhibition of level of core nucleocapsid p24 protein in the supernatant fluid from human peripheral blood mononuclear cells using Retrotek Elisa kit.

It was shown that ULD Ab CD4 inhibited P24 protein by 86±10% when added to a well 24 hours before the infection, a and by 51±3% when added to a well 15 min after the infection of cells with the strain HIV-1LAI. Thus, this experimental model demonstrated the activity of ultra low-doses of rabbit polyclonal antibodies to CD4 (a mixture of homoeopathic dilutions C12+C30+C50) in inhibition of production or amplification of elimination of the protein P24.

What is claimed is:

1. The method of inhibiting of production or amplifying of elimination of P24 protein, said method comprising administering an activated-potentiated form of an antibody to CD4 receptor thereby inhibiting production or amplifying elimination of P24 protein.

2. The method of claim 1, wherein the activated-potentiated form of an antibody to CD4 receptor is to the entire CD4 receptor of SEQ ID NO: 1.

3. The method of claim 1, wherein the activated-potentiated form of an antibody to CD4 receptor is to a fragment of CD4 receptor having sequence selected from group consisting of SEQ ID NO:2, SEQ ID NO:3, SEQ ID NO:4, SEQ ID NO:5, SEQ ID NO:6.

4. The method of claim 1, wherein the activated-potentiated form of an antibody to CD4 receptor is prepared by successive centesimal dilutions coupled with shaking of every dilution.

5. The method of claim 1, wherein the activated-potentiated form of an antibody to CD4 receptor is in the form of mixture of C12, C30, and C50 homeopathic dilutions impregnated onto a solid carrier.

6. The method of claim 1, wherein the activated-potentiated form of an antibody to CD4 receptor is in the form of mixture of C12, C30, and C200 homeopathic dilutions impregnated onto a solid carrier.

7. A pharmaceutical composition for inhibiting of production or amplifying of elimination of P24 protein, comprising an activated-potentiated form of an antibody to CD4 receptor.

8. The pharmaceutical composition of claim 7, wherein the activated-potentiated form of an antibody to CD4 receptor is to the entire CD4 receptor of SEQ ID NO: 1.

9. The pharmaceutical composition of claim 7, wherein the activated-potentiated form of an antibody to CD4 receptor is to a fragment of CD4 receptor having sequences selected from group consisting of SEQ ID NO: 2, SEQ ID NO: 3, SEQ ID NO: 4, SEQ ID NO: 5, SEQ ID NO: 6.

10. The combination pharmaceutical composition of claim 7, wherein the activated-potentiated form of an antibody to CD4 receptor is prepared by successive centesimal dilutions coupled with shaking of every dilution.

11. The combination pharmaceutical composition of claim 7, wherein the activated-potentiated form of an antibody to CD4 receptor is in the form of mixture of C12, C30, and C50 homeopathic dilutions impregnated onto a solid carrier.

12. The combination pharmaceutical composition of claim 7, wherein the activated-potentiated form of an antibody to CD4 receptor is in the form of mixture of C12, C30, and C200 homeopathic dilutions impregnated onto a solid carrier.


SEQUENCE LISTING (6 sequences)

SEQ ID NO: 1 - protein, 458 aa, Homo sapiens
  SOURCE 1..458: /mol_type=“protein” /organism=“Homo sapiens”
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 2 - protein, 434 aa, Homo sapiens
  SOURCE 1..434: /mol_type=“protein” /organism=“Homo sapiens”
GKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 3 - protein, 47 aa, Homo sapiens
  SOURCE 1..47: /mol_type=“protein” /organism=“Homo sapiens”
IGLGIFFCVRCRHRRRQAERMSQIKRLLSEKKTCQCPHRFQKTCSPI

SEQ ID NO: 4 - protein, 36 aa, Homo sapiens
  SOURCE 1..36: /mol_type=“protein” /organism=“Homo sapiens”
GKKVVLGKKGDTVELTCTASQKKSIQFHWKNSNQIK

SEQ ID NO: 5 - protein, 15 aa, Homo sapiens
  SOURCE 1..15: /mol_type=“protein” /organism=“Homo sapiens”
DTYICEVEDQKEEVQ

SEQ ID NO: 6 - protein, 25 aa, Homo sapiens
  SOURCE 1..25: /mol_type=“protein” /organism=“Homo sapiens”
KEEVQLLVFGLTANSDTHLLQGQSL